(19) United States
(12) Patent Application Publication
(10) Pub. No.: US 2020/0107708 A1
(43) Pub. Date: Apr. 9, 2020

(54) ENDOSCOPE

(71) Applicant: OLYMPUS CORPORATION, Tokyo (JP)

(72) Inventor: Keisuke AMANO, Tokyo (JP)

(73) Assignee: OLYMPUS CORPORATION, Tokyo (JP)

(21) Appl. No.: 16/708,716

(22) Filed: Dec. 10, 2019

Related U.S. Application Data
(63) Continuation of application No. PCT/JP2018/019596, filed on May 22, 2018.

(30) Foreign Application Priority Data
Jun. 12, 2017 (JP) 2017-115316

(51) Int. Cl. A61B 1/018; A61B 1/05; A61B 1/00

(52) U.S. Cl. CPC A61B 1/018; A61B 1/00167; A61B 1/00137; A61B 1/05

(57) ABSTRACT

An endoscope includes: an imager that includes a lens and an image transmitter configured to capture an image of a subject; a distal end rigid member that is provided at a distal end part of an insertion portion to be inserted into the subject, the distal end rigid member including a treatment tool insertion path into which a treatment tool to give a given treatment on the subject is inserted, an imaging hole in which the imager is arranged and in which a proximal end side is open, and a groove including a butting face on an end face of the groove on a distal end side; and a butting member arranged in the imager and configured to be butted against the butting face of the groove.

### CROSS-REFERENCE TO RELATED APPLICATION

This application is a continuation of PCT International Application No. PCT/JP2018/019596 filed on May 22, 2018, which designates the United States, incorporated herein by reference, and which claims the benefit of priority from Japanese Patent Application No. 2017-115316, filed on Jun. 12, 2017, incorporated herein by reference.

### BACKGROUND

### 1. Technical Field

The present disclosure relates to an endoscope.

### 2. Related Art

In the field of medicine and the field of industry, endoscopes have been widely used for various examinations. A medical endoscope among them inserts its insertion portion with an imaging unit arranged therein into a body cavity of a subject, such as a patient, thereby enabling acquisition of in-vivo images of the body cavity without incision of the subject. Furthermore, the medical endoscope protrudes its treatment tool from a distal end side in the insertion portion, thereby enabling curative treatment. For these reasons, medical endoscopes have been widely used.

In the endoscope described in Japanese Laid-open Patent Publication No. 2005-261746, an imaging hole for arranging an imaging unit, a treatment tool insertion path, etc., are formed adjacently in a distal end rigid member that is arranged on a distal end side in an insertion portion. In a distal end surface of the distal end rigid member, a treatment tool protrusion port for allowing the treatment tool that is inserted into the treatment tool insertion path to protrude into a subject is formed. Furthermore, an imaging lens of the imaging unit that is arranged in the imaging hole is arranged such that the imaging lens is exposed from the distal end surface of the distal end rigid member.

### SUMMARY

In some embodiments, an endoscope includes: an imager that includes a lens and an image transmitter configured to capture an image of a subject; a distal end rigid member that is provided at a distal end part of an insertion portion to be inserted into the subject, the distal end rigid member including a treatment tool insertion path into which a treatment tool to give a given treatment on the subject is inserted, an imaging hole in which the imager is arranged and in which a proximal end side is open, and a groove including a butting face on an end face of the groove on a distal end side; and a butting member arranged in the imager and configured to be butted against the butting face of the groove.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an endoscope system according to a first embodiment;

FIG. 2 is a perspective view schematically illustrating a configuration of a distal end of an insertion portion of an ultrasound endoscope according to the first embodiment;

FIG. 3 is a partial cross-sectional view schematically illustrating the configuration of the distal end of the insertion portion of the ultrasound endoscope according to the first embodiment;

FIG. 4 is a perspective view illustrating an imaging unit according to the first embodiment;

FIG. 5 is a perspective view of a C-ring that is arranged on the imaging unit;

FIG. 6 is a cross-sectional view of an IG bundle ferrule with the C-ring arranged thereon, viewed from an axial direction of the IG bundle ferrule;

FIG. 7 is an enlarged view of the imaging unit and the vicinity thereof in a distal end rigid member of the ultrasound endoscope according to the first embodiment;

FIG. 8 is an enlarged view of an imaging unit and the vicinity thereof in the distal end rigid member of the ultrasound endoscope according to Comparative Example;

FIG. 9 is a perspective view illustrating another example of the imaging unit;

FIG. 10 is a perspective view of a semi-tubular member that is arranged on the imaging unit;

FIG. 11 is a cross-sectional view of the IG bundle ferrule with the semi-tubular member arranged thereon, viewed from the axial direction of the IG bundle ferrule;

FIG. 12 is an enlarged view of another example of the imaging unit and the vicinity thereof in the distal end rigid member of the ultrasound endoscope according to the first embodiment;

FIG. 13 is a diagram illustrating the case where a prismatic member is arranged as a butting member on an outer circumferential surface of the IG bundle ferrule;

FIG. 14 is a diagram illustrating the case where an oppositely concave member is arranged as a butting member on the outer circumferential surface of the IG bundle ferrule; and

FIG. 15 is an enlarged view of the imaging unit and the vicinity thereof in the distal end rigid member of an ultrasound endoscope according to a second embodiment.

### DETAILED DESCRIPTION

### First Embodiment

A first embodiment of an ultrasound endoscope that is an endoscope to which the present disclosure is applied will be described with reference to the drawings. Note that the present disclosure is not limited by the embodiments described below and, for example, the present disclosure is applicable to an endoscope without an ultrasound transducer. Furthermore, in the drawings, the same parts are denoted with the same reference numbers.

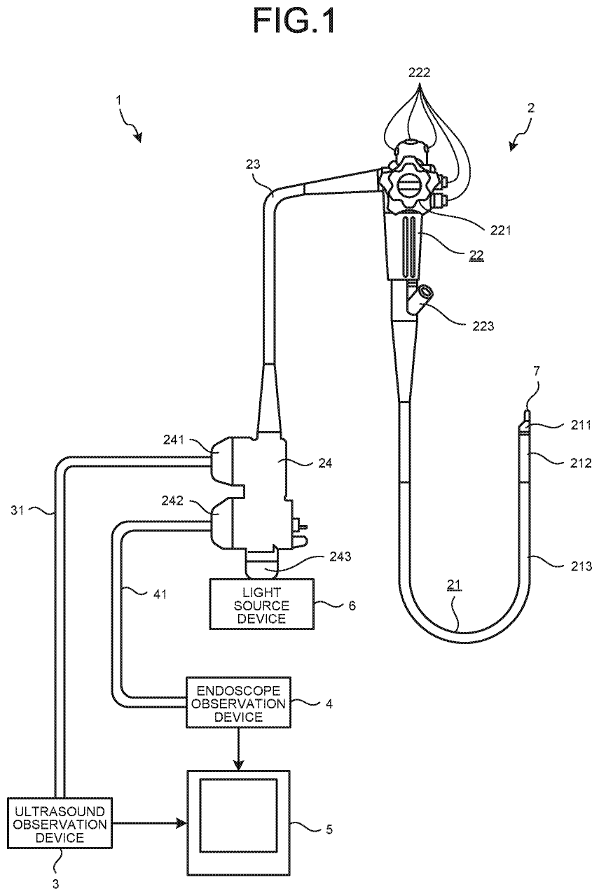

FIG. 1 is a diagram schematically illustrating an endoscope system 1 including an ultrasound endoscope 2 according to a first embodiment. As illustrated in FIG. 1, the endoscope system 1 according to the first embodiment is a system that includes an ultrasound endoscope 2, an ultrasound observation device 3, an endoscope observation device 4, a display device 5, and a light source device 6 and that makes an internal ultrasound diagnosis on a subject, such as a human, using the ultrasound endoscope 2.

Using the ultrasound transducer that is provided at a distal end part of the ultrasound endoscope 2, the ultrasound endoscope 2 converts an electric pulse signal that is received from the ultrasound observation device 3 into an ultrasound pulse (acoustic pulse) and applies the ultrasound pulse to a subject and then converts ultrasound echoes that are reflected on the subject into an electric echo signal that represents the echo signal by a change in voltage and outputs the echo signal.

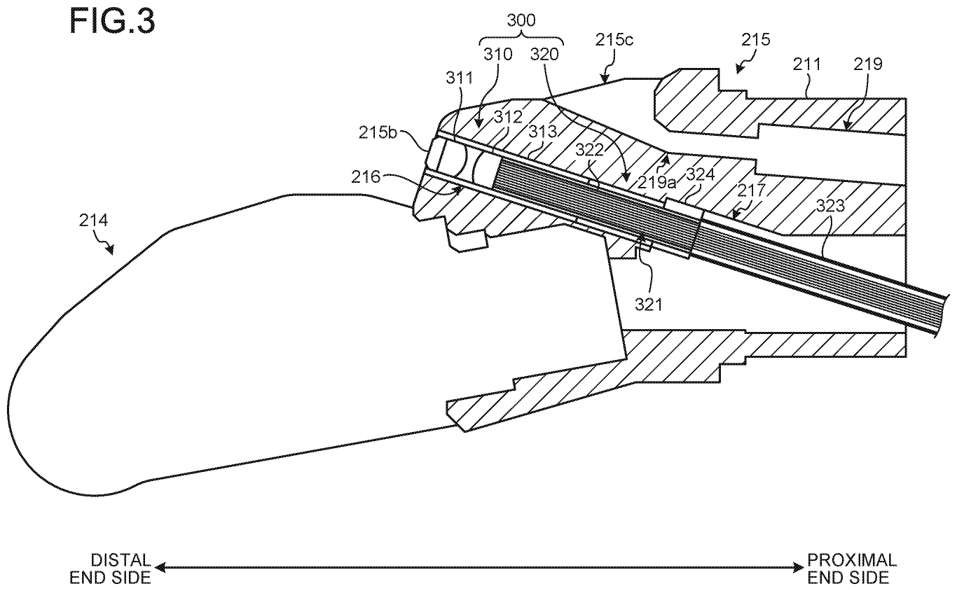

The ultrasound endoscope 2 includes an imaging unit 300 (refer to FIG. 3) that captures images optically and the ultrasound endoscope 2 is inserted into a digestive tract (the esophagus, the stomach, the duodenum or the large intestine) or a respiratory organ (the trachea or a bronchi) of the subject, thereby enabling capturing images of the digestive tract or the respiratory organ. It is also possible to capture images of organs (the pancreas, the gallbladder, a bile duct, the biliary tract, lymph nodes, the organ in the mediastinum, blood vessels, etc.) around the digestive tract or the respiratory organ. The ultrasound endoscope 2 includes a light guide that guides the illumination light that is applied to the subject to capture images using the imaging unit 300. While a distal end part of the light guide reaches the distal end of an insertion portion 21 of the ultrasound endoscope 2 to be inserted into the subject, a proximal end part of the light guide is connected to the light source device 6 that generates illumination light.

As illustrated in FIG. 1, the ultrasound endoscope 2 includes the insertion portion 21, an operation unit 22, an universal cord 23, and a connector 24. The insertion portion 21 is a part that is inserted into the subject. As illustrated in FIG. 1, the insertion portion 21 includes a distal end rigid member 211 that is provided on a distal end side and that supports an ultrasound transducer 7, a curve part 212 that is joined to a proximal end side in the distal end rigid member 211 and a flexible tubular part 213 that is joined to the proximal end side in the curve part 212 and that has flexibility.

The endoscope system 1 according to the first embodiment employs, as the ultrasound transducer 7, a convex type in which a plurality of piezoelectric elements are arranged in an array and electronically switching the piezoelectric elements relating to transmission and reception and delaying transmission and reception by each piezoelectric element enables electronic scanning. The system of the ultrasound transducer 7 is not limited to the convex type, and a radial type or a linear type may be employed.

Although specific illustrations are omitted, in the insertion portion 21, the light guide that transmits the illumination light that is supplied from the light source device 6 and a plurality of signal cables that transmit various signals are arranged and a treatment tool insertion path 219 (see FIG. 3) for inserting a treatment tool, such as forceps, etc., are formed.

The operation unit 22 is a part that is joined to the proximal end side in the insertion portion 21 and that receives various operations from a doctor, or the like. As illustrated in FIG. 1, the operation unit 22 includes a curve knob 221 for operating the curve part 212 such that the curve part 212 curves and a plurality of operation members 222 for performing various operations. In the operation unit 22, a treatment tool insertion port 223 that communicated with the treatment tool insertion path 219 (refer to FIG. 3) and that is for inserting the treatment tool into the treatment tool insertion path 219 is formed.

The universal cord 23 is a cable that extends from the operation unit 22 and in which a plurality of signal cables that transmit various signals and an light guide that transmits the illumination light supplied from the light source device 6, etc., are arranged.

The connector 24 is arranged at the distal end of the universal cord 23 and includes a first connector part 241, a second connector part 242, and a third connector part 243 to which an ultrasound cable 31, a video cable 41, and the light source device 6 are connected, respectively.

The ultrasound observation device 3 is electrically connected to the ultrasound endoscope 2 via the ultrasound cable 31 and outputs a pulse signal to the ultrasound endoscope 2 via the ultrasound cable 31 and the echo signal is input to the ultrasound observation device 3 from the ultrasound endoscope 2. The ultrasound observation device 3 performs given processing on the echo signal to generate an ultrasound image.

The endoscope observation device 4 is electrically connected to the ultrasound endoscope 2 via the video cable 41 and an image signal from the ultrasound endoscope 2 is input to the endoscope observation device 4 via the video cable 41. The endoscope observation device 4 performs given processing on the image signal to generate an endoscopic image.

The display device 5 is formed using liquid crystals or electro luminescence (EL) and displays the ultrasound image that is generated by the ultrasound observation device 3 or the endoscopic image that is generated by the endoscope observation device 4.

The light source device 6 supplies the illumination light to illuminate the inside of the subject to the ultrasound endoscope 2.

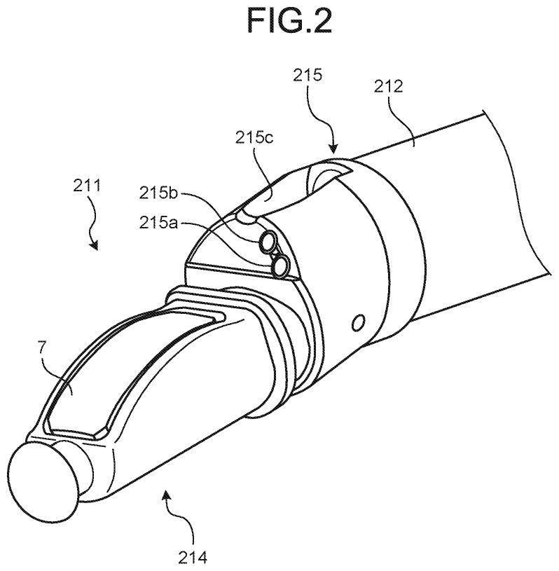

FIG. 2 is a perspective view illustrating a configuration of a distal end of the insertion portion 21 of the ultrasound endoscope 2 according to the first embodiment. FIG. 3 is a partial cross-sectional view illustrating the configuration of the distal end of the insertion portion 21 of the ultrasound endoscope 2 according to the first embodiment. In FIG. 3, illustration of wiring that is connected to an ultrasound transducer module 214 is omitted. As illustrated in FIGS. 2 and 3, the insertion portion 21 includes the ultrasound transducer module 214 that holds the ultrasound transducer 7 and an endoscope module 215 on the distal end side. On a sloping surface of the endoscope module 215 on the distal end side, an illumination lens 215a forming an illumination unit, an observation window 215b forming the imaging unit 300, and a treatment tool protrusion port 215c for allowing a treatment tool, such as forceps, to protrude are provided.

In the distal end rigid member 211 of the endoscope module 215, an imaging hole 216 for arranging the imaging unit 300 is formed obliquely with respect to the longitudinal axial direction of the insertion portion 21. In the distal end rigid member 211, the treatment tool insertion path 219 that communicates with the treatment tool protrusion port 215c is formed such that the end of the treatment tool insertion path 219 communicating with the treatment tool protrusion port 215c and the vicinity thereof are oblique upward to the axial direction of the distal end rigid member 211 and the treatment tool protrudes from the treatment tool protrusion port 215c obliquely upward to the axial direction.

The axial direction of the distal end rigid member 211 herein refers to a direction along the longitudinal direction of the insertion portion 21 extended straightly. Being obliquely upward with respect to the axial direction of the distal end rigid member 211 means the endoscope module 215 in the posture as illustrated in FIGS. 2 and 3 such that the treatment tool insertion path 219 is above the imaging unit 300 and the ultrasound transducer module 214 is positioned under the imaging unit 300.

The imaging unit 300 includes a lens unit 310 and an image guide bundle unit (hereinafter, IG bundle unit) 320 that is an image transmission unit. The image transmission unit may include an imaging device and signal lines, such as a charge coupled device (CCD) or a complementary metal oxide semiconductor (CMOS).

The lens unit 310 may include the observation window 215b and a plurality of lenses, such as objective lenses 311 and 312, that are held by a lens frame 313 and distal ends of image guide fibers (hereinafter, IG fibers) are butted against a surface of the objective lens 312 on the proximal end side. The proximal end side in the lens frame 313 is fixed to the distal end side in an IG bundle ferrule 322 with an adhesive.

The IG bundle unit 320 includes an image guide bundle (hereinafter, IG bundle) 321 obtained by bundling a plurality of IG fibers that are led to the operation unit 22 through the curve part 212 and the flexible tubular part 213; the IG bundle ferrule 322 that is a cylindrical member enclosing the distal end side in the IG bundle 321; and a flexible tube 323 that encloses the IG bundle 321 on the proximal end side with respect to the IG bundle ferrule 322.

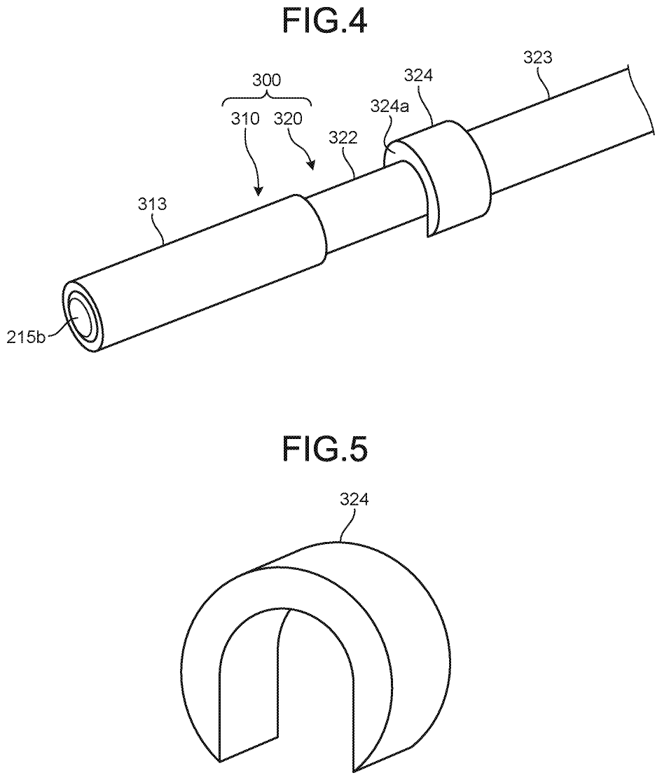

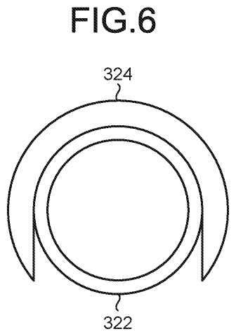

FIG. 4 is a perspective view illustrating the imaging unit 300 according to the first embodiment. FIG. 5 is a perspective view of a C-ring 324 that is arranged on the imaging unit 300. FIG. 6 is a cross-sectional view of the IG bundle ferrule 322 with the C-ring 324 arranged thereon, viewed from an axial direction of the IG bundle ferrule.

In the ultrasound endoscope 2 according to the first embodiment, the C-ring 324 illustrated in FIG. 5 is arranged as a butting member for positioning the imaging unit 300 with respect to the distal end rigid member 211 independently of the IG bundle ferrule 322 on the distal end side in the imaging unit 300 in the distal end rigid member 211, specifically, on an outer circumferential surface of the IG bundle ferrule 322 as illustrated in FIGS. 4 and 6. The C-ring 324 is fixed to the outer circumferential surface of the IG bundle ferrule 322 with an adhesive. The C-ring 324 is formed of metal such as stainless steel, titan, titan alloy, or a Cu—Zn alloy (brass). When the IG bundle ferrule 322 and the C-ring 324 are formed of metal, the C-ring 324 may be fixed to the outer circumferential surface of the IG bundle ferrule 322 not with an adhesive but by welding. The butting member may be arranged on the outer circumferential surface of the IG bundle ferrule 322 integrally with the IG bundle ferrule 322.

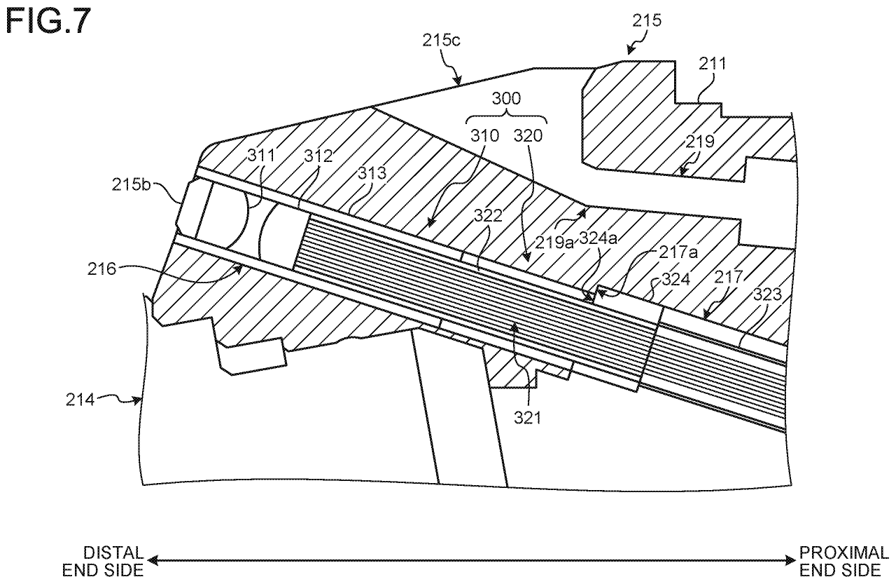

FIG. 7 is an enlarged view of the imaging unit 300 and the vicinity thereof in the distal end rigid member 211 of the ultrasound endoscope 2 according to the first embodiment. In FIG. 7, illustration of wiring that is connected to the ultrasound transducer module 214 is omitted. As illustrated in FIG. 7, a step against which the C-ring 324 arranged on the outer circumferential surface of the IG bundle ferrule 322 is butted is formed on an inner wall surface of the imaging hole 216 on the proximal end side. The step is formed by forming a groove 217 through which the C-ring 324, which is arranged on the outer circumferential surface of the IG bundle ferrule 322, can pass in the inner wall surface of the imaging hole 216 on the proximal end side along the axial direction of the imaging hole 216. An end surface of the groove 217 on the proximal end side is open and thus the end face of the groove 217 on the distal end side serves as a butting face 217a that is butted against an end face 324a of the C-ring 324 on the distal end side. The butting face 217a that is formed in the groove 217 and the end face 324a of the C-ring 324 on the distal end side that is arranged on the IG bundle ferrule 322 are butted against to each other so that the imaging unit 300 is positioned with respect to the distal end rigid member 211.

Positioning the imaging unit 300 with respect to the distal end rigid member 211 in the first embodiment is attaching the imaging unit 300 to the distal end rigid member 211 such that the observation window 215b, which is arranged in the lens unit 310 of the imaging unit 300, is positioned in a given position with respect to the sloping surface of the distal end rigid member 211 on the distal end side such that the observation window 215b is not excessively raised or excessively lowered from the opening of the imaging hole 216 on the distal end side.

A curve part 219a that curves the treatment tool insertion path 219 is formed in the middle of the treatment tool insertion path 219 in the distal end rigid member 211 and the treatment tool insertion path 219 slopes with respect to the imaging hole 216 such that the treatment tool insertion path 219 gets close to the imaging hole 216 from the proximal end side to the curve part 219a and the treatment tool insertion path 219 gets apart from the imaging hole 216 from the curve part 219a to the distal end side (the side of the treatment tool protrusion port 215c). Accordingly, the thickness between the imaging hole 216 and the treatment tool insertion path 219 varies from the distal end side to the proximal end side and the thickness is the smallest in the part where the curve part 219a is positioned.

In the first embodiment, the groove 217 is formed such that the butting face 217a is positioned on the proximal end side with respect to the part with the smallest thickness in the distal end rigid member 211 (the part in which the curve part 219a is positioned) and the C-ring 324 is arranged on the outer circumferential surface of the IG bundle ferrule 322 in alignment with the position of the butting face 217a.

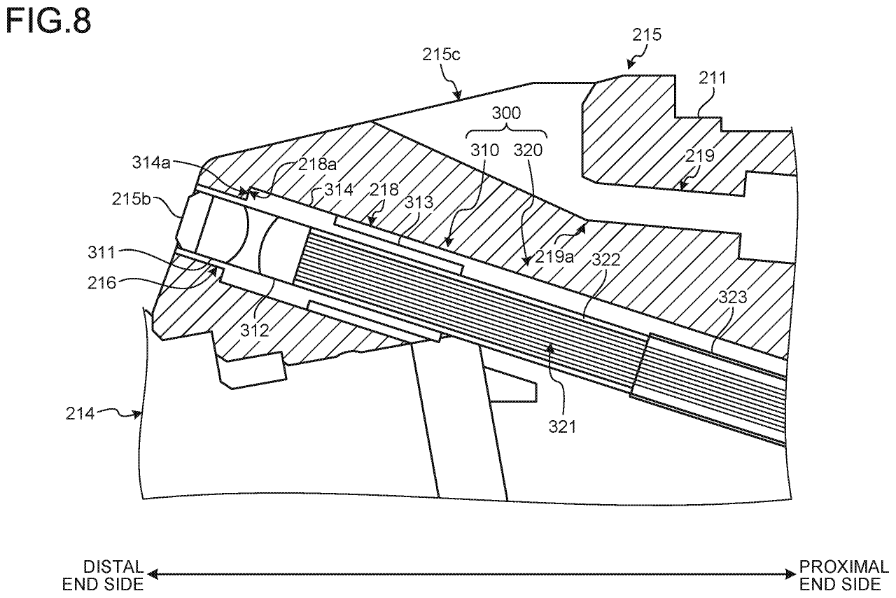

FIG. 8 is an enlarged view of the imaging unit 300 and the vicinity thereof in the distal end rigid member 211 of the ultrasound endoscope 2 according to Comparative Example. In FIG. 8, illustration of the wiring connected to the ultrasound transducer module 214 is omitted. As illustrated in FIG. 8, in Comparative Example, a butting member 314 is formed on the distal end side in the distal end rigid member 211 of the imaging unit 300, specifically, in the lens frame 313 integrally with the lens frame 313. A groove 218 through which the butting member 314 formed in the lens frame 313 can pass when the imaging unit 300 is inserted into the imaging hole 216 from the proximal end side in the distal end rigid member 211 is formed in the inner wall surface of the imaging hole 216 from the proximal end side to the distal end side. A butting face 218a that is an end surface of the groove 218 on the distal end side and an end face 314a of the butting member 314 on the distal end side, which is formed in the lens frame 313, are butted against each other so that the imaging unit 300 is positioned with respect to the distal end rigid member 211.

In Comparative Example, because the groove 218 extends also in the part with the smallest thickness (the part where the curve part 219a is positioned), the thickness in the thinnest part further reduces by the depth of the groove 218 and this results in reduction in durability of the distal end rigid member 211. Furthermore, it is necessary to keep a space for the butting face 218a and the butting member 314 on the distal end side in the distal end rigid member 211, which hinders reduction in diameter of the distal end rigid member 211 on the distal end side.

On the contrary, in the ultrasound endoscope 2 according to the first embodiment, the groove 217 does not extend to the part with the smallest thickness (the part in which the curve part 219a of the treatment tool insertion path 219 is positioned) and this enables inhibition of reduction in durability of the distal end rigid member 211 due to excessive reduction in thickness. A space for the butting face 217a and the C-ring 324 is preferably kept on the proximal end side in the distal end rigid member 211 and thus it is possible to position the imaging unit 300 with respect to the distal end rigid member 211 while reducing the diameter of the distal end rigid member 211 on the distal end side.

In the ultrasound endoscope 2 according to the first embodiment, the groove 217 is formed in the circumferential direction along almost the whole circumference of the inner wall surface of the imaging hole 216 on the proximal end side. In the first embodiment, when the imaging unit 300 is attached to the distal end rigid member 211, the imaging unit 300 is rotated in the imaging hole 216 for adjusting the resolution resulting from the direction in which each IG fiber of the IG bundle unit 320 is aligned. Thus, formation of the groove 217 in the circumferential direction along almost the whole circumference of the inner wall surface enables formation of the butting face 217a along almost the whole circumference of the inner wall surface in the circumferential direction, which makes it possible to assuredly butt the end face 324a of the C-ring 324 on the distal end side against the butting face 217a to position the imaging unit 300 with respect to the distal end rigid member 211 regardless in which direction the C-ring faces along with rotation of the imaging unit 300 after adjustment for a given resolution is made.

Assembling the imaging unit 300 will be described next. In the first embodiment, first of all, the observation window 215b and the objective lens 311 are arranged on the end of the lens frame 313 on the distal end side. The IG bundle 321 is then arranged in the IG bundle ferrule 322 such that the opening of the IG bundle ferrule 322 on the distal end side and the distal ends of the IG fibers forming the IG bundle 321 are flush with each other and the IG bundle ferrule 322 and the IG bundle 321 are fixed with an adhesive, or the like. Note that, the flexible tube 323 is previously arranged for the IG bundle 321 excluding the part on the distal end side where the IG bundle ferrule 322 is arranged. The surface of the objective lens 312 forming the lens unit 310 on the proximal end side and the distal ends of the IG fibers that are arranged in the IG bundle ferrule 322 are brought into contact with each other and the objective lens 312 is fixed to the end face of the IG bundle ferrule 322 on the distal end side with an adhesive, or the like. The distal end part of the IG bundle unit 320 to which the objective lens 312 is fixed is then inserted into the lens frame 313 from the opening of the lens frame on the proximal end side. The IG bundle unit 320 is then moved back and forth in the lens frame to adjust the distance between the objective lens 311 and the objective lens 312 and, after optical adjustment, such as focus adjustment, is performed, the IG bundle ferrule 322 is adhered and fixed to the lens frame 313 by an adhesive, or the like. Accordingly, assembling the imaging unit 300 completes with the lens unit 310 and the IG bundle unit 320 being integrated with each other.

After the imaging unit 300 is assembled as described above, the C-ring 324 is arranged by being fixed to the outer circumferential surface of the IG bundle ferrule 322 with an adhesive, or the like, with a given distance from the distal end of the lens unit 310 being kept. The imaging unit 300 is inserted into the imaging hole 216 from the proximal end side in the distal end rigid member 211 and the end face 324a of the C-ring 324 on the distal end side is butted against the butting face 217a via the groove 217 that is formed in the inner wall surface of the imaging hole 216 on the proximal end side to position the imaging unit 300 with respect to the distal end rigid member 211.

After the imaging unit 300 is positioned with respect to the distal end rigid member 211 as described above, the imaging unit 300 is rotated in the imaging hole 216 for adjusting the resolution resulting from the direction in which each IG fiber of the IG bundle unit 320 is aligned. Fixing the imaging unit 300 to the distal end rigid member 211 with an adhesive, or the like, after the resolution is adjusted completes attaching the imaging unit 300 to the distal end rigid member 211.

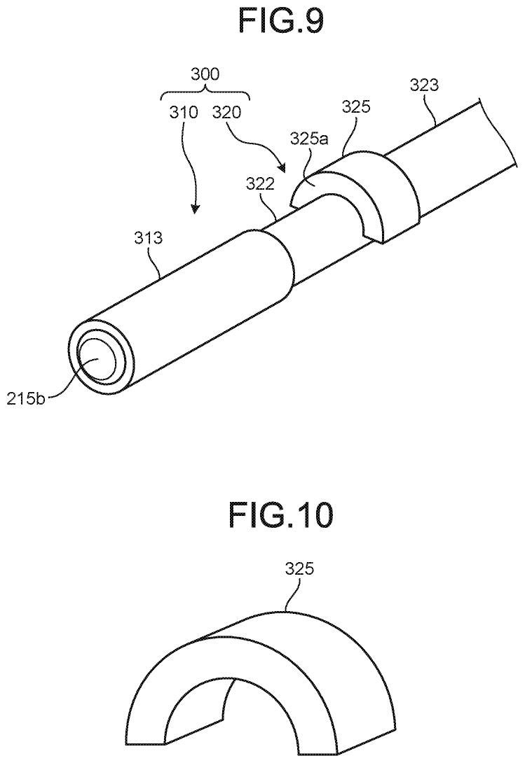

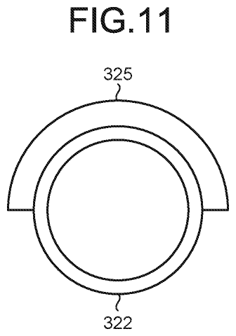

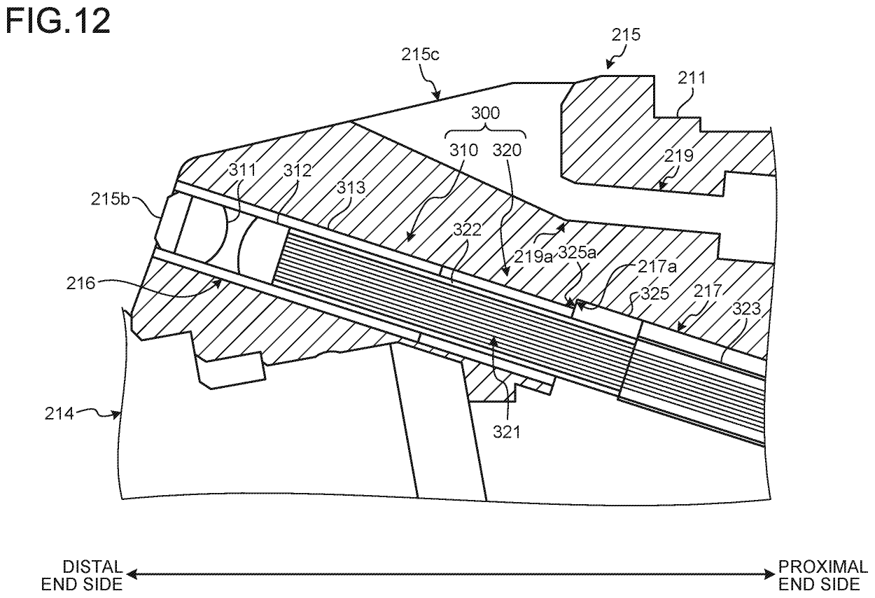

FIG. 9 is a perspective view illustrating another example of the imaging unit 300. FIG. 10 is a perspective view of a semi-tubular member 325 that is arranged in the imaging unit 300. FIG. 11 is a cross-sectional view of the IG bundle ferrule 322 with the semi-tubular member 325 arranged thereon, viewed from the axial direction of the IG bundle ferrule. FIG. 12 is an enlarged view of another example of the imaging unit 300 and the vicinity thereof in the distal end rigid member 211 of the ultrasound endoscope 2 according to the first embodiment. In FIG. 12, illustration of the wiring connected to the ultrasound transducer module 214 is omitted.

In the imaging unit 300 illustrated in FIG. 9, instead of the C-ring 324, the semi-tubular member 325 illustrated in FIG. 10 is used as the butting member that is arranged on the IG bundle ferrule 322. As illustrated in FIG. 11, the semi-tubular member 325 is arranged independently from the IG bundle ferrule 322 on the outer circumferential surface of the IG bundle ferrule 322 and the IG bundle ferrule 322 and the semi-tubular member 325 are fixed to each other with an adhesive. As illustrated in FIG. 12, an end face 325a of the semi-tubular member 325 on the distal end side is butted against the butting face 217a of the imaging hole 216 to position the imaging unit 300 with respect to the distal end rigid member 211.

When the semi-tubular member 325 is arranged on the IG bundle ferrule 322 as the butting member of the imaging unit 300, the groove 217 may be formed in the circumferential direction along almost the whole circumference of the inner wall surface of the imaging hole 216 on the proximal end side or the groove 217 may be formed in a semi-circular shape only on the side of the treatment tool insertion path 219 in the circumferential direction on the inner wall surface. On the other hand, when the groove 217 in the semi-circular shape is formed, before the imaging unit 300 is inserted into the imaging hole 216, the imaging unit 300 is rotated about the axis of the imaging unit 300 to adjust the resolution resulting from the direction in which each IG fiber of the IG bundle unit 320 is aligned. The semi-tubular member 325 is then attached to the IG bundle ferrule 322 with the posture of the imaging unit 300 after the resolution adjustment being kept and the imaging unit 300 is inserted into the imaging hole 216 from the proximal end side in the distal end rigid member 211. Accordingly, it is possible to, while ensuring a desired resolution, position the imaging unit 300 with respect to the distal end rigid member 211 by butting the end face 325a of the semi-tubular member 325 on the distal end side against the butting face 217a of the imaging hole 216.

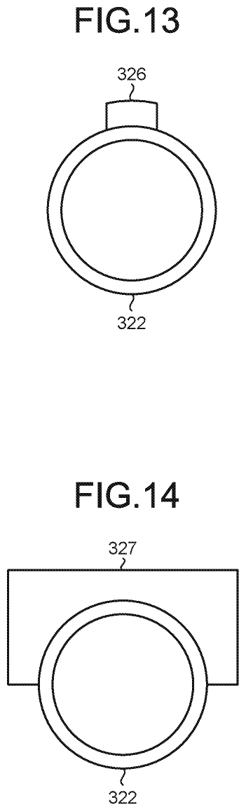

FIG. 13 is a diagram illustrating the case where a prismatic member 326 is arranged as a butting member on an outer circumferential surface of the IG bundle ferrule 322. FIG. 14 is a diagram illustrating the case where an oppositely concave member 327 is arranged as a butting member on the outer circumferential surface of the IG bundle ferrule 322. The butting member that is arranged on the imaging unit 300 on the proximal end side may be formed by arranging, on the outer circumferential surface of the IG bundle ferrule 322 illustrated in FIG. 14 and independently of the IG bundle ferrule 322, the prismatic member 326 illustrated in FIG. 13 or the oppositely concave member 327 that is illustrated in FIG. 17 and that is concave on the side where the oppositely concave member 327 makes contact with the outer circumferential surface of the IG bundle ferrule 322.

### Second Embodiment

A second embodiment of an endoscope to which the present disclosure is applied will be described below with reference to the drawings. In the illustration of the drawings, the same parts as those of the above-described first embodiment are denoted with the same reference numbers.

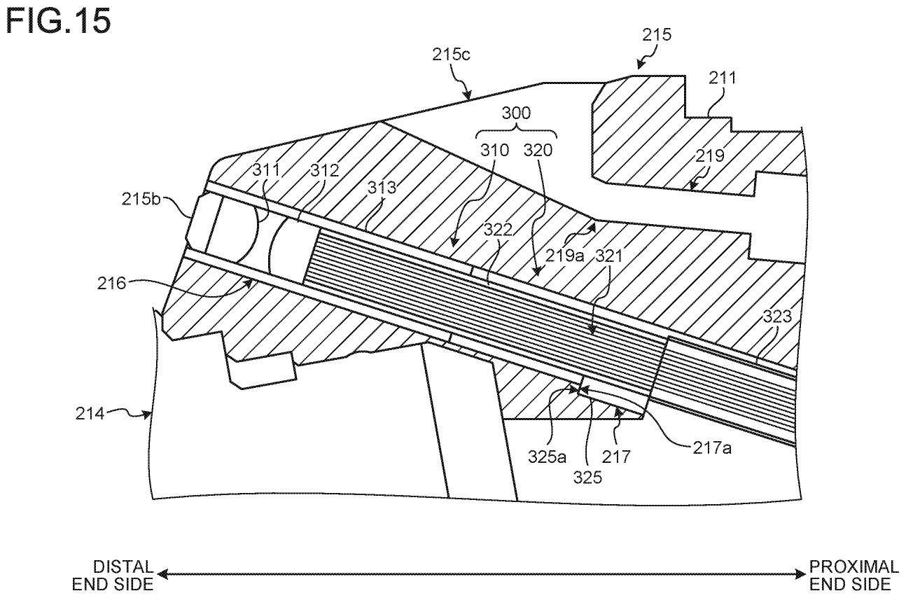

FIG. 15 is an enlarged view of the imaging unit 300 and the vicinity thereof in the distal end rigid member 211 of an ultrasound endoscope according to the second embodiment. In FIG. 15, illustration of the wiring that is connected to the ultrasound transducer module 214 is omitted. As illustrated in FIG. 15, as the groove 217 that is formed in the inner wall surface of the imaging hole 216 on the proximal end side in the distal end rigid member 211, the groove 217 may be formed not on the inner wall surface on the side of the treatment tool insertion path 219 but in a semi-circular shape in the inner wall surface on the side opposite to the treatment tool insertion path 219. Furthermore, in this case, as the butting member that can pass through the groove 217 in the semi-circular shape, the semi-tubular member 325 independent of the IG bundle ferrule 322 is arranged on and fixed to the outer circumferential surface of the IG bundle ferrule 322 with an adhesive. As illustrated in FIG. 15, the imaging unit 300 is inserted into the imaging hole 216 from the proximal end side in the distal end rigid member 211 and the end face 325a of the semi-tubular member 325 is butted against the butting face 217a of the imaging hole 216 to position the imaging unit 300 with respect to the distal end rigid member 211.

This enables a larger thickness between the imaging hole 216 and the treatment tool insertion path 219 in the distal end rigid member 211 than that obtained when the groove 217 is formed in the inner wall surface on the side of the treatment tool insertion path 219 and thus enable increase in durability of the distal end rigid member 211. Furthermore, a space for the butting face 217a and the semi-tubular member 325 is preferably kept on the proximal end side in the distal end rigid member 211 and thus it is possible to position the imaging unit 300 with respect to the distal end rigid member 211 while reducing the diameter of the distal end rigid member 211 on the distal end side.

When the imaging unit 300 is attached to the distal end rigid member 211, the imaging unit 300 is rotated about its axial line before the imaging unit 300 is inserted into the imaging hole 216 to adjust the resolution resulting from the direction in which each IG fiber of the IG bundle unit 320 is aligned. The semi-tubular member 325 is attached to the IG bundle ferrule 322 with the posture of the imaging unit 300 after the resolution adjustment being kept and the imaging unit 300 is then inserted into the imaging hole 216 from the distal end side in the distal end rigid member 211. This makes it possible to, while ensuring a desired resolution, position the imaging unit 300 with respect to the distal end rigid member 211 by butting the end face 325a of the semi-tubular member 325 on the distal end side against the butting face 217a of the imaging hole 216.

The endoscope according to the present disclosure is useful for positioning the imaging unit with respect to the distal end rigid member while reducing the diameter of the distal end rigid member on the distal end side.

The endoscope according to the present disclosure fulfills an effect that it is possible to position the imaging unit with respect to the distal end rigid member while reducing the diameter of the distal end rigid member on the distal end side.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an imager that includes a lens and an image transmitter configured to capture an image of a subject;
a distal end rigid member that is provided at a distal end part of an insertion portion to be inserted into the subject, the distal end rigid member including
a treatment tool insertion path into which a treatment tool to give a given treatment on the subject is inserted,
an imaging hole in which the imager is arranged and in which a proximal end side is open, and
a groove including a butting face on an end face of the groove on a distal end side; and
a butting member arranged in the imager and configured to be butted against the butting face of the groove.

2. The endoscope according to claim 1, wherein an inner wall surface of the groove includes a step that is butted against an end face of the butting member as the butting face.

3. The endoscope according to claim 1, wherein the image transmitter includes an image transmission member and a ferrule that is arranged on a distal end side of the transmission member, and
the butting member is arranged independently of the ferrule on an outer circumferential surface of the ferrule.

4. The endoscope according to claim 3, wherein the butting member is a C-ring.

5. The endoscope according to claim 4, wherein the C-ring is fixed to the ferrule by adhesion.

6. The endoscope according to claim 3, wherein the image transmission member includes an image guide bundle obtained by bundling a plurality of image guide fibers or includes an imaging sensor and a signal line.

7. The endoscope according to claim 1, wherein the distal end rigid member has a thickness between the imaging hole and the treatment tool insertion path varying in an axial direction of the distal end rigid member from a distal end side to a proximal end side and the end face of the groove is positioned closer to the proximal end side of the distal end rigid member than a part where the thickness is the smallest.
